(19) United States
(12) Patent Application Publication
(10) Pub. No.: US 2015/0159220 A1
(43) Pub. Date: Jun. 11, 2015

(54) METHODS FOR PREDICTING AND DETECTING CANCER RISK

(71) Applicant: Fred Hutchinson Cancer Research Center, Seattle, WA (US)

(72) Inventors: Brian J. Reid, Seattle, WA (US); Xiaohong Li, Redmond, WA (US)

(21) Appl. No.: 14/400,522

(22) PCT Filed: May 10, 2013
(86) PCT No.: PCT/US2013/040653

§ 371 Date: Nov. 11, 2014

Related U.S. Application Data
(60) Provisional application No. 61/646,096, filed on May 11, 2012.
(60) Provisional application No. 61/774,854, filed on Mar. 8, 2013.

(51) Int. Cl. C12Q 1/68; G06F 19/00; G06F 19/22

(52) U.S. Cl. CPC C12Q 1/6886; G06F 19/22; G06F 19/3431; C12Q 2600/156; C12Q 2600/16; C12Q 2600/118

(57) ABSTRACT

Disclosed herein are methods for predicting and detecting cancer risk using genetic markers such as somatic genomic alterations (SGA) that are associated with cancer risk. Also disclosed herein are methods for predicting and detecting a risk of esophageal adenocarcinoma (EA) based on the use of SGA that are associated with a risk of EA.

### GOVERNMENT FUNDING

Work described herein was supported, at least in part, under NIH Challenge Grant #1RC1CA146973, and NIH P01 Grant #2P01CA0951955. The U.S. government, therefore, may have certain rights in this invention.

### TECHNICAL FIELD

This disclosure relates to methods for predicting and detecting cancer risk using genetic markers such as somatic genomic alterations (SGA) that are indicative of cancer risk. More specifically, this disclosure relates to methods for predicting and detecting a risk of esophageal adenocarcinoma (EA) based on the use of SGA that are associated with a risk of EA.

### BRIEF DESCRIPTION OF THE DRAWINGS

FIG. 1 is a Kaplan-Meier (KM) curve plot showing 5-year progression of EA in risk-stratified patients initially assigned to high (top line), medium (middle line) and low risk (bottom line) based on the sample biopsy SGA analysis data and cancer risk prediction model.

FIG. 2 is a KM curve plot showing the progression to EA of patients initially assigned to the medium risk group and then reassigned to high (top line), medium (middle line) and low risk (bottom line) using data from a second endoscopy.

FIG. 3 is a KM curve plot based on sample biopsy SGA analysis data showing the three EA risk groups sample biopsy data collected over a 250-month interval starting from baseline assessment (or first biopsy). The cancer risk prediction model was used to stratify subjects into 3 risk groups, high (top line), medium (middle line) and low risk (bottom line).

### DETAILED DESCRIPTION

Disclosed herein are methods for predicting and detecting cancer risk in a subject. In particular embodiments, the methods disclosed herein may be used to predict and/or detect a risk of EA in a subject. The methods disclosed herein comprise the analysis of a sample from a subject for the presence or absence of certain biomarkers including SGA, and further methods of developing a cancer risk prediction model for calculating a risk score for predicting and detecting the risk of EA in the subject. In certain embodiments, the prediction and/or detection of the risk of EA in subject may be used to recommend treatment or prevention strategies or predict the likely outcome of the disease. In other embodiments, the methods disclosed herein may allow for assessing, classifying, and/or stratifying individuals at risk of EA, or individuals diagnosed with EA, into different risk subgroups.

### DEFINITIONS

The term “somatic genomic alteration,” or SGA, refers to DNA sequence changes or aberrations that have accumulated in the genome of a cell during the lifetime of a subject. SGA include point mutations, deletions, gene fusions, gene amplifications, translocations, copy number gain, copy number loss, copy-neutral loss of heterozygosity, homozygous deletion, and chromosomal rearrangements. In some cases these mutations are benign and do not progress to disease during a normal life time, however in other cases they may lead to diseases such as cancer.

The term “copy number,” refers to the DNA copy number at one or more genetic loci. The copy number measurement can assess if a sample has any genomic copy number alterations, i.e., containing amplifications and deletions of genetic loci. Amplifications and deletions can affect a part of a genetic element, an entire element, or many elements simultaneously. A copy number analysis does not necessarily determine the exact number of amplifications or deletions, but identifies those regions that contain the genetic alterations, and whether the alteration is a deletion or amplification compared to a subject's constitutive genome. In some embodiments, a copy number can be measured in a subject's healthy, normal cells, and compared to the same subject's suspected or targeted diseased cells.

The terms “copy number variation” or CNV (in germline cells), and “copy number alteration” or CNA (in somatic cells), as used herein, refer to structural genetic variations including additions or deletions in the number of copies of a particular segment of DNA when compared to a reference genome sequence. The term copy gain refers to sections of a chromosome that demonstrate a gain, an addition, or duplication of DNA compared to a subject's constitutive genome. A copy gain may be an allele-specific copy gain wherein a specific allele is amplified or duplicated. A copy gain may also include a balanced copy gain which indicate a region of a chromosome or a whole chromosome that duplicated maternal and paternal chromosomes with equal numbers. The term copy loss refers to sections of a chromosome that demonstrate a loss or deletion of DNA compared to a subject's constitutive genome. A copy loss may be an allele-specific copy loss wherein a specific allele is deleted.

The term “copy-neutral loss of heterozygosity,” or cnLOH, or alternatively uniparental disomy, refers to loss of heterozygosity caused by duplication of a maternal (unimaternal) or paternal (unipaternal) chromosome or chromosomal region and concurrent loss of the other allele. In certain instances, cnLOH may have an acquired, clonal derivation, caused by early mitotic errors and autozygosity. Alternatively, cnLOH may have a constitutional, nonclonal derivation when an organism receives two copies of a chromosome, or part of a chromosome, from one parent and no copies from the other parent due to errors in meiosis I or meiosis II. This loss of heterozygosity may result in a non-functional allele. The term homozygous deletion (HD) refers to a deletion of both copies of the same allele or the same chromosomal segment of a pair of homologous chromosomes.

A nucleic acid array (“array”) comprises nucleic acid probes attached to a solid support. Arrays typically comprise a plurality of different nucleic acid probes that are coupled to a surface of a substrate in different, known locations. These arrays, also described as a SNP array, DNA microarray, DNA chip, biochip, etc., have been generally described in the art. For example, these arrays can generally be produced using mechanical synthesis methods or light directed synthesis methods that incorporate a combination of photolithographic methods and solid phase synthesis methods. Although a planar array surface may be used, the array can be fabricated on a surface of virtually any shape or even a multiplicity of surfaces. Arrays can be nucleic acids on beads, gels, polymeric surfaces, and fibers such as fiber optics, glass or any other appropriate substrate. In certain embodiments, arrays can be designed to cover an entire genome using single nucleotide polymorphisms (SNPs).

A “probe” is a surface-immobilized molecule that can be recognized by a particular target. In certain embodiments, a probe refers to an oligonucleotide designed for use in connection with a SNP microarray or any other microarrays known in the art that are capable of selectively hybridizing to at least a portion of a target sequence under appropriate conditions. In general, a probe sequence is identified as being either complementary (i.e., complementary to the coding or sense strand (+)), or reverse complementary (i.e., complementary to the anti-sense strand (−)). Probes can have a length of about 10-100 nucleotides, or about 15-75 nucleotides, and alternatively from about 15-50 nucleotides.

The term “hybridization” refers to the formation of complexes between nucleic acid sequences, which are sufficiently complementary to form complexes via Watson-Crick base pairing or non-canonical base pairing. For example, when a primer “hybridizes” with a target sequence (template), such complexes (or hybrids) are sufficiently stable to serve the priming function required by, e.g., the DNA polymerase, to initiate DNA synthesis. Hybridizing sequences need not have perfect complementarity to provide stable hybrids. In many situations, stable hybrids form where fewer than about 10% of the bases are mismatches. As used herein, the term “complementary” refers to an oligonucleotide that forms a stable duplex with its complement under assay conditions, generally where there is about 80%, about 81%, about 82%, about 83%, about 84%, about 85%, about 86%, about 87%, about 88%, about 89%, about 90%, about 91%, about 92%, about 93%, about 94% about 95%, about 96%, about 97%, about 98% or about 99% greater homology. Those skilled in the art understand how to estimate and adjust the stringency of hybridization conditions such that sequences having at least a desired level of complementarity stably hybridize, while those having lower complementarity will not. Examples of hybridization conditions and parameters are well-known (Ausubel, 1987; Sambrook and Russell, 2001).

The terms “labeled” and “labeled with a detectable label” are used interchangeably and specify that an entity (e.g., a fragment of DNA, a primer or a probe) can be visualized, for example following binding to another entity (e.g., an amplification product). The detectable label can be selected such that the label generates a signal that can be measured and which intensity is related to (e.g., proportional to) the amount of bound entity. A wide variety of systems for labeling and/or detecting nucleic acid molecules, such as primer and probes, are well-known. Labeled nucleic acids can be prepared by incorporating or conjugating a label that is directly or indirectly detectable by spectroscopic, photochemical, biochemical, immunochemical, electrical, optical, and chemical or other means. Suitable detectable agents include radionuclides, fluorophores, chemiluminescent agents, microparticles, enzymes, colorimetric labels, magnetic labels, haptens and the like.

The term “subject” or “patient” encompasses mammals and non-mammals. Examples of mammals include: humans, other primates, such as chimpanzees, and other apes and monkey species; farm animals such as cattle, horses, sheep, goats, swine; domestic animals such as rabbits, dogs, and cats; laboratory animals including rodents, such as rats, mice and guinea pigs. Examples of non-mammals include birds and fish.

The terms “treat,” “treating” and “treatment,” mean alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “linkage disequilibrium” refers to the non-random association of alleles at two or more loci.

### SGA Analysis

The methods disclosed herein provide for the detection or prediction of cancer risk based on the presence or absence or one or more SGA. The SGA used for the methods disclosed herein include, for example, CNA such as copy gain and copy loss, as well as cnLOH and HD. Generally, the somatic genomes of many Barrett's esophagus sufferers have some SGA and most individuals who do not progress to EA largely maintain genomic integrity over prolonged periods of time, typically without high levels of cnLOH or large chromosomal gains and losses. However, those who progress to cancer may develop significantly increased SGA, increased heterogeneity and highly correlated chromosomal events involving large portions of the genome associated with risk of progression to EA.

The SGA disclosed herein may range in size from a single nucleotide to a DNA segment including part or all of a chromosome. In certain embodiments, the SGA disclosed herein may range from 1 kilobase (kb) up to one or more megabases (Mb) in size, including large chromosomal regions. The SGA used for the methods disclosed herein may be located on one or more chromosomes.

The SGA analysis described herein may be performed by methods known by those of skill in the art. For example, SGA analysis may be performed using DNA sequencing based technologies such as whole genome DNA sequencing or by DNA sequencing of certain parts of a genome such as one or more particular chromosomes or specific chromosomal locations or regions. Additional methods for SGA analysis may include the use of DNA microarray, SNP array, DNA chip, biochip, array comparative genome hybridization (aCGH), and other microarray technologies. Furthermore, SGA analysis may be performed using genetic markers such as single nucleotide polymorphisms (SNP), restriction fragment length polymorphisms (RFLP), microsatellite markers, simple sequence repeat (SSR), simple sequence length polymorphisms (SSLP), amplified fragment length polymorphism (AFLP), random amplification of polymorphic DNA (RAPD), variable number tandem repeat (VNTR), etc. The genetic markers used for the methods disclosed herein may be dominant or co-dominant markers.

The methods disclosed herein include the use of one or more genetic samples obtained from a subject for SGA analysis. In certain embodiments, the genetic samples may include, e.g., a biological fluid or tissue. Examples of biological fluids include, e.g., whole blood, serum, plasma, cerebrospinal fluid, urine, tears or saliva. Examples of tissue include, e.g., connective tissue, muscle tissue, nervous tissue, epithelial tissue, and combinations thereof. In particular embodiments, the genetic sample may be provided from tumor or cancer tissues. In other embodiments, the genetic sample may be provided from pre-cancerous tissues. In such embodiments, the genetic samples may be provided from a subject having the premalignant condition Barrett's esophagus, a precursor of EA. In further embodiments, the genetic sample may be provided from a control or reference tissue. The control or reference sample may be a normal healthy tissue sample or paired normal healthy tissue sample from the same subject as the tumor or cancer tissue sample. In one embodiment, the genetic sample may be a tissue biopsy from the esophagus of a subject with Barrett's Esophagus paired with a blood or gastric sample from the same subject.

After obtaining the genetic sample for use with the methods disclosed herein, genomic DNA may be extracted from the samples according to standard practices such as, for example, phenol-chloroform extraction, salting out, digestion-free extraction or by the use of commercially available kits, such as the DNEasy® or QIAAMP® kits (Qiagen, Valencia, Calif.). The DNA obtained from the samples can then be modified or altered to facilitate analysis.

The isolated DNA may be amplified using routine methods. Useful nucleic acid amplification methods include the Polymerase Chain Reaction (PCR) and variations of PCR including TAQMAN®-based assays and reverse transcriptase polymerase chain reaction (RT-PCR). The resulting amplified DNA may be purified, using routine techniques, such as MINELUTE® 96 UF PCR Purification system (Qiagen). After purification, the amplified DNA can be fragmented using sonication or enzymatic digestion, such as DNase I. After fragmentation, the DNA may be labeled with a detectable label.

In particular embodiments of the methods disclosed herein, once the amplified, fragmented DNA is labeled with a detectable label, it can be hybridized to a microarray. The microarray may contain oligonucleotides, genes or genomic clones that can be used in SGA analysis as disclosed herein. For example, the microarray can contain oligonucleotides or genomic clones that detect mutations or polymorphisms, such as single nucleotide polymorphisms (SNPs). In particular embodiments, SGA analysis may be performed using a SNP genotyping array or microarray. A SNP genotyping array may be used for whole-genome or targeted SGA analysis. Microarrays can be made using routine techniques known in the art. Alternatively, commercially available microarrays can be used. Examples of microarrays that can be used are the Illumina Omni Quad 1M SNP array (Illumina Inc., San Diego, Calif.), AFFYMETRIX® GENECHIP® Mapping 100K Set SNP Array (Affymetrix, Inc., Santa Clara, Calif.), the Agilent Human Genome aCGH Microarray 44B (Agilent Technologies, Inc., Santa Clara, Calif.), Nimblegen aCGH microarrays (Nimblegen, Inc., Madison, Wis.), etc. Reviews regarding the operation of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Ghee et al. (1996) “Accessing Genetic Information with High-Density DNA Arrays.” Science 274:610-614, each of which is incorporated herein by reference.

After hybridization, the microarray may be washed to remove non-hybridized nucleic acids. In some embodiments, after washing, the microarray is analyzed in a reader or scanner. Examples of readers and scanners include GENECHIP® Scanner 3000 G7 (Affymetrix, Inc.), the Agilent DNA Microarray Scanner (Agilent Technologies, Inc.), GENEPIX® 4000B (Molecular Devices, Sunnyvale, Calif.), etc. Signals gathered from the probes contained in the microarray can be analyzed using commercially available software, such as those provided by Illumina, Affymetrix or Agilent Technologies. For example, if the GENECHIP® Scanner 3000 G7 from Affymetrix is used, the AFFYMETRIX® GENECHIP® Operating Software can be used. The AFFYMETRIX® GENECHIP® Operating Software collects and extracts the raw or feature data (signals) from the AFFYMETRIX® GENECHIP® scanners that detect the signals from all the probes. The data collected from the microarray may be used to determine the presence or absence of SGA at one or more loci on the chromosomal DNA provided in the genetic samples. Furthermore, the results of the microarray analysis may be used to identify SGAs that are associated with the risk of cancer.

The methods disclosed herein to predict and/or detect cancer risk include the analysis of a genetic sample for the presence or absence of one or more SGA having a significant association with the risk of cancer. In specific embodiments, the methods disclosed herein may include the analysis of a specific chromosomal locus or chromosomal region having one or more SGA selected from at least one of copy gain, copy loss, allele specific copy loss, allele specific copy gain, cnLOH, balanced gain, and HD. The power of the methods disclosed herein to detect cancer risk may be improved by using a panel or combination of two or more chromosomal loci or regions located on one or more chromosomes, wherein each chromosomal loci or region includes one or more SGA selected from at least one of copy gain, copy loss, allele specific copy loss, allele specific copy gain, cnLOH, balanced gain, and HD. In such embodiments, the chromosomal regions to be examined for the presences or absence of certain SGA may comprise from 1, 2 chromosomal regions up to 100 chromosomal regions, or more. For example, the panel of chromosomal regions may include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chromosomal regions that may be examined for the presences or absence of certain SGA such as copy gain, copy loss, cnLOH, balanced gain, and HD, or combinations thereof.

The SGA used according to the methods disclosed herein may be found at one or more chromosomal regions of the human genome. The human chromosomal regions disclosed herein may range in size from approximately 1 nucleotide to 100 kb, from 1 nucleotide to 1 Mb, from 1 nucleotide to 100 Mb, and from 1 nucleotide up to and including an entire chromosome. The SGA disclosed herein may be found at locations on the short or long arms of one or more of the 23 pairs of chromosomes (22 pairs of autosomes and one pair of sex chromosomes) in a human cell. For example, the SGA disclosed herein may be found on one or more of human chromosome 1, 2, 3, 4, 5, 6, 7, 8, 9, 10, 11, 12, 13, 14, 15, 16, 17, 18, 19, 20, 21, 22 and sex chromosomes X and Y.

The chromosomal regions described herein may be identified or labeled according to their chromosomal location, chromosomal interval, cytogenetic map location, chromosome sequence map, gene location, etc. In certain embodiments, a positive score or result for the presence of an SGA in a particular chromosomal region may be determined by identifying one or more SGA in the chromosomal region. In other embodiments, a positive score or result for the presence of an SGA in a particular chromosomal region may be determined by identifying one or more specific SGA in at least one approximately 1 Mb segment of the chromosomal region of interest. The presence of a specific SGA may be scored or reported as a “yes” or a “1” and the absence of the specific SGA may be scored or reported as a “no” or a “0”.

The chromosomal regions and SGA described herein that are associated with an increased risk of cancer may be identified according to methods known in the art for genetic association studies. In some embodiments, genetic variation or polymorphism at one or more genetic markers, such as one or more SGA biomarkers, may be predictive of whether an individual is at risk or susceptible to disease, such as EA. For example, the association of one or more genetic markers with a disease phenotype may be identified by the use of a genome wide association study (GWAS). As generally known by those of skill in the art, a GWAS is an examination of genetic polymorphism across the entire genome and is designed to identify genetic polymorphisms that are associated with a trait, phenotype, or disease of interest. For example, if certain genetic polymorphisms are more or less frequent in individuals with a disease of interest, the genetic variations can be said to be “associated” with the disease. Generally, the polymorphisms associated with the disease may directly cause the disease and/or they may be in linkage disequilibrium with one or more genetic regions or elements that may influence the disease or a risk of disease.

In some embodiments, the genetic markers, such as SGA biomarkers, that may be associated with an increased risk of cancer can be identified using a case-control study to find those SGA that can be used to separate and distinguish disease progressors from non-progressors. Statistical analysis may be used to identify a panel or combination of SGA biomarkers that are significantly associated with an increased risk of cancer, and one or more statistical methods or operations such as, for purposes of example only, sequential forward selection with bootstrap method, Cox proportional-hazards regression model, backwards and forwards stepwise selection, and area under the ROC (Receiver operating characteristic) curve (AUC) may be used to identify individual SGA and/or panels or combinations of SGA associated with cancer risk.

The statistical analysis of SGA in genetic samples from a case-control or case-cohort study may identify chromosomal regions having SGA biomarkers associated with a risk of EA. In one such embodiment, the statistical analysis of SGA from genetic samples using, for example, sequential forward selection with bootstrap method, may be used to identify relatively large chromosomal regions, or mega regions, having SGA associated with a risk or EA, such as least one of cnLOH SGA on chromosome 13 between chromosome location 20-115 Mb; copy number gain SGA on chromosome 15 between chromosome location 20-103 Mb; copy number gain SGA on chromosome 17 between chromosome location 25-81 Mb; copy number loss SGA on chromosome 17 between chromosome location 0-23 Mb; cnLOH SGA on chromosome 17 between chromosome location 0-23 Mb; and copy number gain SGA on chromosome 18 between chromosome location 0-36 Mb.

### Risk Prediction Model

The methods disclosed herein for detecting or predicting cancer risk in a subject may comprise a risk prediction model based on the SGA analysis of certain combinations of the chromosomal regions disclosed herein. The scored results from the SGA analysis of a panel of chromosomal regions disclosed herein may be further examined by statistical analysis and then combined and grouped into a set of cancer risk prediction features that may be used for detecting or predicting cancer risk. In particular embodiments, the panel of chromosomal regions each comprise SGA types selected from one or more of copy gain, copy loss, cnLOH, balanced gain, and HD, or combinations thereof, wherein any combination of all or part of the scored results of the panel of chromosomal regions may then be combined and/or added together to provide a set of risk prediction features that may be used for detecting or predicting cancer risk.

In certain embodiments, the sum of the results from the SGA analysis of the panel of chromosomal regions may be combined with the results of the SGA analysis of one or more subsets of the panel of chromosomal regions. In one such embodiment, a method of predicting and detecting cancer risk may comprise a set of risk prediction features including the sum of the SGA analysis results from a panel of one or more chromosomal regions which may then be combined with one or more of the sum of the results of the copy gain SGA from the chromosomal regions, the sum of the results of the HD SGA from the chromosomal regions, the sum of the results of the cnLOH SGA from the chromosomal regions, the sum of the results of the copy loss SGA from the chromosomal regions, or the sum of the balanced gain SGA from the chromosomal regions. In another such embodiment, presented here for purposes of example only, a method of predicting and detecting cancer risk may comprise a set of risk prediction features including the sum of the SGA analysis results from a panel of approximately 86 chromosomal regions, approximately 1 Mb each, which may then be combined with one or more of the sum of the results of the copy gain SGA from the 86 chromosomal regions, the sum of the results of the HD SGA from the 86 chromosomal regions, the sum of the results of the cnLOH SGA from the 86 chromosomal regions, the sum of the results of the copy loss SGA from the 86 chromosomal regions, the sum of the allele specific copy gain from the 86 chromosomal regions, the sum of the allele specific copy loss from the 86 chromosomal regions, and the sum of the balanced gain SGA from the 86 chromosomal regions.

The results of the SGA analysis of a primary panel of one or more chromosomal regions may be examined statistically in order to select subsets or groupings of SGA that may be analyzed, alone or together, with the results from the primary panel of chromosomal regions to predict and/or determine a risk of cancer in a subject. In some embodiments, a statistical examination such as a combined sequential forward selection and AUC may be performed on the SGA analysis results from a panel of chromosomal regions, wherein the results of the statistical analysis are used to select a set of risk prediction features for predicting and/or detecting cancer risk. In such embodiments, a statistical examination of the results of a SGA analysis of a panel of chromosomal regions, may be used to select a set of approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more risk prediction features for predicting and/or detecting cancer risk. In a specific embodiment, presented here for purposes of example only, a statistical examination of a panel of approximately 86 chromosomal regions, approximately 1 Mb each, may be used to select a set of approximately 29 risk prediction features. In a specific example of such an embodiment, a statistical examination of a panel of 86 chromosomal regions, 1 Mb each, may be used to select a set of 29 risk prediction features, wherein the set of 29 risk prediction features may be as follows: (1) the allele specific copy gain SGA on chromosome 6 at chromosome location 1-2 Mb; (2) the allele specific copy gain SGA on chromosome 15 at chromosome location 70-71 Mb; (3) the allele specific copy gain SGA on chromosome 17 at chromosome location 37-38 Mb; (4) the allele specific copy gain SGA on chromosome 18 at chromosome location 19-20 Mb; (5) the homozygous deletion SGA on chromosome 2 at chromosome location 226-227 Mb; (6) the cnLOH SGA on chromosome 6 at chromosome location 29-30 Mb; (7) the cnLOH SGA on chromosome 6 at chromosome location 146-147 Mb; (8) the cnLOH SGA on chromosome 7 at chromosome location 78-79 Mb; (9) the cnLOH SGA on chromosome 8 at chromosome location 138-139 Mb; (10) the cnLOH SGA on chromosome 11 at chromosome location 38-39 Mb; (11) the cnLOH SGA on chromosome 11 at chromosome location 110-111 Mb; (12) the cnLOH SGA on chromosome 13 at chromosome location 42-43 Mb; (13) the cnLOH SGA on chromosome 17 at chromosome location 9-10 Mb; (14) the cnLOH SGA on chromosome 17 at chromosome location 12-13 Mb; (15) the cnLOH SGA on chromosome 19 at chromosome location 48-49 Mb; (16) the allele specific copy loss SGA on chromosome 1 at chromosome location 36-37 Mb; (17) the allele specific copy loss SGA on chromosome 9 at chromosome location 0-1 Mb; (18) the allele specific copy loss SGA on chromosome 9 at chromosome location 33-34 Mb; (19) the allele specific copy loss SGA on chromosome 17 at chromosome location 8-9 Mb; (20) the allele specific copy loss SGA on the 9 chromosome at chromosome location 65-66 Mb; (21) the allele specific copy loss SGA on the X chromosome at chromosome location 42-43 Mb; (22) the allele specific copy loss SGA on the Y chromosome at chromosome location 13-14 Mb; (23) the sum of the results of all the copy loss SGA from the 86 chromosomal regions; and (24) the sum of all the SGA analysis results from the panel of 86 chromosomal regions. (25) the allele specific copy gain SGA on chromosome 6 at chromosome location 5-6; (26) the cnLOH SGA on chromosome 5 at chromosome location 93-94; (27) the cnLOH SGA on chromosome 11 at chromosome location 50-51; (28) the allele specific copy loss SGA on chromosome 7 at chromosome location 77-78; (29) the allele specific copy loss SGA on chromosome 12 at chromosome location 45-46.

The methods of predicting and/or detecting the risk of cancer in a subject may include the development of a cancer risk prediction model comprising the steps of (1) obtaining a paired sample (one representing normal DNA, and one from a targeted tissue or organ, i.e. esophagus) or use targeted organ only from the subject: (2) analyzing the sample for the presence or absence of SGA in of a panel of the 86 chromosomal regions; (3) and then using the SGA analysis results from the panel of 86 chromosomal regions to select a set of risk prediction features as disclosed herein. In certain embodiments, the methods disclosed herein may comprise the development of a prediction model wherein the set of risk prediction features as described herein may be weighted according to their importance or value to the prediction model. For example, the weight assigned to each of the risk prediction features may be designed to be proportional to the predictive power that it should have in predicting EA risk in a subject. In such embodiments, the weights for each of the set of risk prediction features may be a positive or negative coefficient calculated according to methods known to those of skill in the art such as, for example, a logistic regression model, neural networks, discrimination analysis, vector support machine, and other models for classification.

The SGA analysis results from a panel of chromosome regions and/or the selected set of risk prediction features may then be used to develop a prediction model to calculate a cancer risk score. In certain embodiments, a cancer risk score (s) may be calculated using the formula (1):

s=β₀+Σᵢ₌₁ⁿβᵢxᵢ (1)

Where xᵢ are the set of risk prediction features from 1 to n (x₁, x₂, x₃ . . . xₙ), βᵢ is the weighted value assigned to the risk prediction feature xᵢ (i runs from 1 to n, and n is the number of risk prediction features in the set of risk prediction features). The calculated risk score (s) may be normalized to a range between 0 and 1 by setting β₀ to −3.108 and then calculating the scaled risk prediction score using formula (2):

1/(1+e^{-s}) (2).

Where e is Euler's number defined in mathematics, and approximately e=2.718281828.

The methods disclosed herein include the calculation of a normalized risk score with continuous values ranging from 0 to 1 which may be used to predict or detect a risk of cancer, such as EA, in a subject. For example, the normalized risk score of approximately 0.50 or greater may be considered a risk score indicating a high risk of EA in a subject. In such embodiments, a normalized risk score of approximately 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, and 1.0 may be considered a risk score indicating a high risk of EA in a subject. In other embodiments, a normalized risk score ranging from approximately 0.05 to approximately 0.49 may be considered a risk score indicating a medium risk of EA in a subject. In such embodiments, a normalized risk score of approximately 0.05, 0.1, 0.15, 0.2, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, and 0.49 may be considered a risk score indicating a medium risk of EA in a subject. In yet further embodiments, a normalized risk score ranging from approximately 0.0 to approximately 0.05 may be considered a risk score indicating a low risk of EA in a subject. In such embodiments, a normalized risk score of approximately 0.01, 0.02, 0.03, 0.04, 0.05, 0.06, 0.07, 0.08, 0.09, 0.10, 0.11, 0.12, 0.13, 0.14, and 0.049 may be considered a risk score indicating a low risk of EA in a subject. In still other embodiments, one or more risk groups may be used to stratify subjects according to their risk score. For example, 1, 2, 3, 4, 5, 6, 7, 8, 9, 10, or more, risk groups might be used to stratify subjects according to their risk score. In another example, subjects may be stratified into one or more of “high risk”, “intermediate-high risk”, “medium risk”, “intermediate-low risk”, and “low risk” groups according to their EA risk scores.

In some embodiments, the methods for predicting and/or detecting cancer risk in a subject may include the following steps: (1) obtaining a genetic sample from a subject; (2) determining the presence or absence of one or more SGA in at least one chromosomal region from the genetic sample; (3) selecting at least one risk prediction from the at least one chromosomal region; and (5) providing a cancer risk score based on the at least one risk prediction feature, wherein the cancer risk score is predictive of the cancer risk in the subject. The step of selecting a set of risk prediction features may be based on the SGA analysis results of the panel of chromosomal regions and may also comprise determining or calculating weight values for each of the set of risk prediction features, wherein the weight values are used in the step of providing a cancer risk score.

In other embodiments, the methods for predicting and/or detecting cancer risk in a subject may include the following steps: (1) obtaining a genetic sample from a subject; (2) isolating chromosomal DNA from the sample; (3) determining the presence or absence of one or more SGA in a panel of chromosomal regions from the chromosomal DNA of the sample; (4) selecting a set of risk prediction features based on the SGA analysis results; and (5) providing a cancer risk score based on the set of risk prediction features by (a) weighting the set of risk prediction features, (b) calculating the cancer risk score using formula (1), and (c) normalizing the cancer risk score using formula (2), wherein a cancer risk score of approximately 0.50 or greater is indicative of a high cancer risk in the subject, wherein a cancer risk score of approximately 0.05 to approximately 0.49 is indicative of a medium cancer risk in the subject, and wherein a cancer risk score of approximately 0.0 to approximately 0.049 is indicative of a low cancer risk in the subject.

In yet further embodiments, the methods for predicting and/or detecting EA cancer risk in a subject may include the following steps: (1) obtaining a genetic sample from a subject at risk of EA; (2) isolating chromosomal DNA from the sample; (3) determining the presence or absence of one or more SGA in a panel of 86 chromosomal regions from the chromosomal DNA of the sample; (4) selecting a set of 29 risk prediction features based on the SGA analysis results of the panel of 86 chromosomal regions; and (5) calculating a cancer risk score based on the set of 29 risk prediction features by (a) weighting the set of 29 risk prediction features, (b) calculating the cancer risk score using formula (1), and (c) normalizing the cancer risk score using formula (2), wherein a cancer risk score of approximately 0.50 or greater is indicative of a high cancer risk in the subject, wherein a cancer risk score of approximately 0.05 to approximately 0.49 is indicative of a medium cancer risk in the subject, and wherein a cancer risk score of approximately 0.0 to approximately 0.049 is indicative of a low cancer risk in the subject.

Also provided herein are kits for practicing the methods disclosed herein. In certain embodiments, the kit may include a carrier for the various components of the kit. In certain such embodiments, the carrier can be a container or support, in the form of, e.g., bag, box, tube, rack, and is optionally compartmentalized. The carrier may define an enclosed confinement for safety purposes during shipment and storage. In particular embodiments, the kits for use with the methods disclosed herein may include various components useful in collecting and preparing genetic samples, and in predicting and/or determining cancer risk according to the current disclosure. For example, the kit many include oligonucleotides, probes, DNA microarrays, or SNP arrays, syringes, scalpels and related reagents and materials useful in predicting and/or determining cancer risk according to the current disclosure. In some embodiments, the kit comprises reagents (e.g., probes, primers, and or antibodies) for determining the presence or absence of one or more SGA as disclosed herein.

The oligonucleotides and probes in the kits as disclosed herein can be labeled with any suitable detection marker including but not limited to, radioactive isotopes, fluorophores, biotin, enzymes (e.g., alkaline phosphatase), enzyme substrates, ligands and antibodies, etc. Alternatively, the oligonucleotides and probes included in the kits disclosed herein are not labeled, and instead, one or more markers are provided in the kit so that users may label the oligonucleotides and probes at the time of use.

In certain embodiments, various other compositions and components may be provided in the kits disclosed herein including, but not limited to, Taq polymerase, deoxyribonucleotides, dideoxyribonucleotides, other primers suitable for the amplification of a target DNA sequence, RNase A, and the like. In particular embodiments, the kits disclosed herein may include instructions on using the kit for the methods disclosed herein.

### EXAMPLES

### Example 1

A longitudinal case-cohort study was performed on esophageal epithelium biopsy samples collected over 25 years from 248 subjects. The case-cohort study was designed to characterize the development of SGA in cells of the esophagus and to predict the risk of EA in subjects. A total of 1272 biopsies were examined, including 473 biopsies from 79 participants with Barrett's esophagus (BE) who progressed to cancer during follow-up (“progressors”) and 799 biopsies from 169 participants with BE who did not progress to cancer within the follow-up time (“non-progressors”) (Table 1).

TABLE 1

| Gender | Non-progressor | Progressor |
|---|---|---|
| male | 140 | 71 |
| female | 29 | 8 |
| Total | 169 | 79 |
| Age (mean range) | 60.9 (30.4-83.6) | 64.7 (35.4-83.7) |

Each of the biopsy samples were paired with normal constitutive controls comprising blood or gastric tissue samples from the same subject. The paired samples were analyzed using Illumina Omni quad 1M SNP arrays (Illumina Inc., San Diego, Calif.) according to standard methods to characterize SGA, including copy gain, copy loss, cnLOH, and HD. Use of a paired constitutive control allowed detection of cnLOH and the ability to distinguish between gain of both parental alleles (balanced gains) and allele-specific gains. Appendix A shows the results of the genome-wide SNP array analysis which identified SGA biomarkers having a significant association with a risk of developing EA. More specifically, Appendix A shows the identification of certain types of SGA biomarkers located in 1 Mb genomic segments at specified chromosome locations. Also shown in Appendix A is the frequency of each of the identified SGA biomarkers in progressors and non-progressors and the hazard ratio.

### Example 2

After the genome-wide SNP array analysis and the identification of SGA biomarkers associated with a risk of developing EA (Appendix A), additional analysis was performed to select a specific panel of SGA biomarkers that could be used for EA risk prediction. Using sequential forward selection with bootstrap method, 6 genomic regions (“mega regions”) shown in Table 2 were identified as EA predictors.

TABLE 2

| Mega | | | Chromosomal |
|---|---|---|---|
| Region | SGA type | Chromosome | Location |
| 1 | cnLOH | 13 | 20 Mb to 115 Mb |
| 2 | gain | 15 | 20 Mb to 103 Mb |
| 3 | gain | 17 | 25 Mb to 81 Mb |
| 4 | loss | 17 | 0 Mb to 23 Mb |
| 5 | cnLOH | 17 | 0 Mb to 23 Mb |
| 6 | gain | 18 | 0 Mb to 36 Mb |

The SNP probes used for the SNP array analysis and identification of the mega regions listed in Table 2 are provided in Appendix B. Each of the entries in Appendix B includes the Illumina SNP identifiers used in the Illumina Omni-Quad 1M SNP arrays (Illumina, Inc. San Diego, Calif.).

The scores for the presence or absence of the specified SGA marker in any 1 Mb segment of any one of the 6 mega regions were used to successfully separate EA progressors from EA non-progressors. For mega region 1, the average binary correlation between each 1 Mb position in this region measured by cosine index (0=no match, 1=perfect matched or linked) was 0.931. For mega region 2, the average binary correlation between each 1 Mb position in this region measured by cosine index is 0.793. For mega region 3, the average binary correlation between each 1 Mb position in this region measured by cosine index was 0.784. For mega region 4, the average binary correlation between each 1 Mb position in this region measured by cosine index was 0.950. For mega region 5, the average binary correlation between each 1 Mb position in this region measured by cosine index was 0.911. For mega region 6, the average binary correlation between each 1 Mb position in this region measured by cosine index was 0.772. The combination of the specified SGA biomarker scores of any 1 Mb segment from across all 6 of the mega regions would further improve EA risk prediction and separation of EA progressors from EA non-progressors.

### Example 3

Further examination of the results from the genome-wide SNP array analysis and the SGA biomarkers from Example 1 (Appendix A), identified additional 1 Mb genomic regions with SGA biomarkers for EA risk prediction. The 1 Mb regions were identified using stepwise regression, including backward elimination and forward stepwise selection for the type of SGA biomarker (gain, loss, cnLOH, balanced gain, and HD) listed for each 1 Mb genomic region listed in Appendix A. The regression analysis initially selected 231 chromosomal regions that were then combined using Cox proportional hazards regression model to identify 86 chromosomal regions of 1 Mb that are significant predictors of EA risk. As shown in Table 3, the 86 chromosomal regions are identified by SGA biomarkers including 17 copy gain, 4 HD, 39 cnLOH, 23 copy loss, and 3 balanced gain. The SNP probes used for the SNP array analysis and identification of the 86 chromosomal regions listed in Table 3 are provided in Appendix C. Each of the entries in Appendix C includes SNP identifiers as used in the Illumina Omni-Quad 1M SNP arrays (Illumina, Inc. San Diego, Calif.).

TABLE 3

| | | Location |
|---|---|---|
| SGA type | Chromosome | (Mb) |
| Allele specific copy gain | 1 | 9-10 |
| Allele specific copy gain | 3 | 178-179 |
| Allele specific copy gain | 6 | 1-2 |
| Allele specific copy gain | 6 | 5-6 |
| Allele specific copy gain | 8 | 32-33 |
| Allele specific copy gain | 8 | 33-34 |
| Allele specific copy gain | 9 | 16-17 |
| Allele specific copy gain | 9 | 72-73 |
| Allele specific copy gain | 9 | 74-75 |
| Allele specific copy gain | 12 | 57-58 |
| Allele specific copy gain | 13 | 44-45 |
| Allele specific copy gain | 13 | 71-72 |
| Allele specific copy gain | 15 | 70-71 |
| Allele specific copy gain | 17 | 37-38 |
| Allele specific copy gain | 18 | 19-20 |
| Allele specific copy gain | 18 | 30-31 |
| Allele specific copy gain | Y | 3-4 |
| Homozygous deletion | 6 | 1-2 |
| Homozygous deletion | 9 | 10-11 |
| Homozygous deletion | 20 | 15-16 |
| Homozygous deletion | 21 | 36-37 |
| cnLOH | 1 | 21-22 |
| cnLOH | 1 | 39-40 |
| cnLOH | 2 | 202-203 |
| cnLOH | 2 | 208-209 |
| cnLOH | 2 | 226-227 |
| cnLOH | 3 | 41-42 |
| cnLOH | 4 | 46-47 |
| cnLOH | 4 | 160-161 |
| cnLOH | 5 | 84-85 |
| cnLOH | 5 | 91-92 |
| cnLOH | 5 | 93-94 |
| cnLOH | 5 | 121-122 |
| cnLOH | 5 | 136-137 |
| cnLOH | 5 | 170-171 |
| cnLOH | 6 | 29-30 |
| cnLOH | 6 | 62-63 |
| cnLOH | 6 | 146-147 |
| cnLOH | 7 | 66-67 |
| cnLOH | 7 | 78-79 |
| cnLOH | 8 | 138-139 |
| cnLOH | 9 | 134-135 |
| cnLOH | 10 | 22-23 |
| cnLOH | 10 | 61-62 |
| cnLOH | 11 | 18-19 |
| cnLOH | 11 | 38-39 |
| cnLOH | 11 | 50-51 |
| cnLOH | 11 | 110-111 |
| cnLOH | 12 | 6-7 |
| cnLOH | 13 | 42-43 |
| cnLOH | 13 | 58-59 |
| cnLOH | 14 | 61-62 |
| cnLOH | 15 | 88-89 |
| cnLOH | 16 | 61-62 |
| cnLOH | 16 | 76-77 |
| cnLOH | 17 | 9-10 |
| cnLOH | 17 | 12-13 |
| cnLOH | 19 | 33-34 |
| cnLOH | 19 | 34-35 |
| cnLOH | 19 | 48-49 |
| Allele specific copy loss | 1 | 36-37 |
| Allele specific copy loss | 1 | 93-94 |
| Allele specific copy loss | 3 | 60-61 |
| Allele specific copy loss | 4 | 3-4 |
| Allele specific copy loss | 4 | 40-41 |
| Allele specific copy loss | 6 | 62-63 |
| Allele specific copy loss | 7 | 77-78 |
| Allele specific copy loss | 8 | 127-128, |
| Allele specific copy loss | 9 | 0-1 |
| Allele specific copy loss | 9 | 8-9 |
| Allele specific copy loss | 9 | 33-34 |
| Allele specific copy loss | 9 | 35-36 |
| Allele specific copy loss | 9 | 38-39 |
| Allele specific copy loss | 9 | 65-66 |
| Allele specific copy loss | 10 | 25-26 |
| Allele specific copy loss | 10 | 77-78 |
| Allele specific copy loss | 12 | 45-46 |
| Allele specific copy loss | 17 | 8-9 |
| Allele specific copy loss | X | 7-8 |
| Allele specific copy loss | X | 42-43 |
| Allele specific copy loss | X | 154-155 |
| Allele specific copy loss | Y | 8-9 |
| Allele specific copy loss | Y | 13-14 |
| Balanced gain | 1 | 19-20 |
| Balanced gain | 4 | 167-168 |
| Balanced gain | 11 | 55-56 |

Included in the 1 Mb regions listed in Table 3 are multiple 1 Mb genomic regions from the 6 mega regions identified in Example 2, Table 2. More specifically, the 8 overlapping 1 Mb regions from Example 2, and listed in Table 3, are (1) the copy gain from chromosome 15 (70-71 Mb), (2) copy gain from chromosome 17 (37-38 Mb), (3 & 4) copy gain from chromosome 18 (19-20 Mb, 30-31 Mb), (5 & 6) cnLOH from chromosome 13 (42-43 Mb, 58-59 Mb), (7) cnLOH from chromosome 17 (9-10 Mb), and (8) copy loss from chromosome 17 (8-9 Mb). It should be noted that, for the 1 Mb regions listed in Table 3 that include 1 Mb regions from the 6 mega regions from Example 2, any 1 Mb region from the indicated mega region may be used and scored for the appropriate SGA biomarker type listed in Table 3. It should also be noted that scores for the presence or absence of one or more of the SGA biomarkers for the 1 Mb regions listed in Table 3 may be used individually or combined in any combination to produce a score for EA risk prediction in a patient.

### Example 4

The 86 chromosomal regions described in Example 3 (Table 3) were used to develop an EA risk prediction model. For each of the 86 chromosomal regions and their associated SGA listed in Table 3, the presence of the specified SGA in the 1 Mb region is scored as a yes (or 1 in the risk model), and the absence of the specified SGA in the 1 Mb region is scored as a no (or 0 in the risk model). As part of the development of the EA risk prediction model, the SGA analysis scores of the 86 chromosomal regions were grouped into 6 subsets as follows:

1. The sum of the SGA scores of the 17 copy gain regions.

2. The sum of the SGA scores of the 4 HD regions.

3. The sum of the SGA scores of the 39 cnLOH regions.

4. The sum of the SGA scores of the 23 copy loss regions.

5. The sum of the SGA scores of the 3 balanced gain regions.

6. The sum of the SGA scores from all of the 86 regions.

The 6 subsets of SGA analysis scores were used to determine the EA risk prediction model by sequential forward selection and area under the curve (AUC) comparison to arrive at a set of 29 EA risk prediction features shown in Table 4.

TABLE 4

| Risk | | | Chromosome |
|---|---|---|---|
| Prediction | | | Location |
| Feature | SGA type | Chr. # | (Mb) |
| 1 | Allele specific copy gain | 6 | 1-2 |
| 2 | Allele specific copy gain | 6 | 5-6 |
| 3 | Allele specific copy gain | 15 | 70-71 |
| 4 | Allele specific copy gain | 17 | 37-38 |
| 5 | Allele specific copy gain | 18 | 19-20 |
| 6 | cnLOH | 2 | 226-227 |
| 7 | cnLOH | 5 | 93-94 |
| 8 | cnLOH | 6 | 29-30 |
| 9 | cnLOH | 6 | 146-147 |
| 10 | cnLOH | 7 | 78-79 |
| 11 | cnLOH | 8 | 138-139 |
| 12 | cnLOH | 11 | 38-39 |
| 13 | cnLOH | 11 | 50-51 |
| 14 | cnLOH | 11 | 110-111 |
| 15 | cnLOH | 13 | 42-43 |
| 16 | cnLOH | 17 | 9-10 |
| 17 | cnLOH | 17 | 12-13 |
| 18 | cnLOH | 19 | 48-49 |
| 19 | Allele specific copy Loss | 1 | 36-37 |
| 20 | Allele specific copy Loss | 7 | 77-78 |
| 21 | Allele specific copy Loss | 9 | 0-1 |
| 22 | Allele specific copy Loss | 9 | 33-34 |
| 23 | Allele specific copy Loss | 9 | 65-66 |
| 24 | Allele specific copy Loss | 12 | 45-46 |
| 25 | Allele specific copy Loss | 17 | 8-9 |
| 26 | Allele specific copy Loss | X | 42-43 |
| 27 | Allele specific copy Loss | Y | 13-14 |
| 28 | Sum of copy Loss listed | see | see Table 3 |
| | in Table 3 | Table 3 | |
| 29 | Sum of all 86 SGA listed | see | see Table 3 |
| | in Table 3 | Table 3 | |

### Example 5

Based on the SGA analysis results of the 86 chromosomal regions in Table 3, and the 29 risk prediction features listed in Table 4, an EA risk prediction model was developed to calculate an EA risk score (s). The EA risk score was calculated for the 248 samples from Example 1 using the formula (3):

s=β₀+Σᵢ₌₁²⁹βᵢxᵢ (1)

In formula (3), xᵢ are the set of risk prediction features from 1 to 29 (x₁, x₂, x₃ . . . x₂₉), and βᵢ is the weighted value assigned to the risk prediction feature xᵢ. The calculated risk score (s) was normalized to a range between 0 and 1 by setting β₀ to −3.108 and then calculating the scaled risk prediction score using formula (2).

Using a logistic regression model, a weight value βᵢ was calculated for each of the set of 29 risk prediction features listed in Table 4. The assigned weight values were designed to be proportional to the predictive power that each risk prediction feature should have for predicting EA risk in a subject. The calculated mean weight combinations for the 29 risk prediction features, as numbered in Table 4, are as follows: (1) 71.533, (2) 38.664, (3) 11.86, (4) 31.81, (5) 0.82257, (6) 54.66, (7) 63.287, (8) 2.0625, (9) 24.666, (10) 101.06, (11) 79.646, (12) 61.317, (13) −291.97, (14) 12.137, (15) 23.348, (16) −70.412, (17) 99.209, (18) 47.058, (19) 109.08, (20) 68.945, (21) −2.394, (22) 1.649, (23) −27.847, (24) 6.6363, (25) −0.078246, (26) 86.339, (27) 1.9427, (28) −0.033952, and (29) 0.11415.

In addition to the logistic regression model, weight values for the set of 29 risk prediction features were also alternatively calculated by building neural networks with one hidden layer in which two neurons were used in the hidden layer. Log-sigmoid function and linear transfer function were used for hidden layer and output layer of the neural networks respectively. Good prediction results were achieved using this method with 10% of the samples used for cross validation. The weights of the set of 29 risk prediction features (inputs) to hidden neuron 1 were: (1) −11.153; (2) −7.6398; (3) −6.9938; (4) −9.7388; (5) −1.2524; (6) −13.124; (7) −10.043; (8) −4.1967; (9) −5.3834; (10) −13.087; (11) −8.4635; (12) −8.1262; (13) 54.03; (14) −2.203; (15) −4.8398; (16) 6.3942; (17) −14.2; (18) 17.261; (19) −16.149; (20) −8.1497; (21) 3.5944; (22) −8.2795; (23) 4.9805; (24) −7.2584; (25) −0.46784; (26) −16.588; (27) −7.0684; (28) 23.145; and (29) −1.695.

The weights of the set of 29 risk prediction features (inputs) to hidden neuron 2 were: (1) 0.80936, (2) −10.883, (3) 4.7946, (4) 7.7267, (5) −8.5495, (6) −13.122, (7) 8.0354, (8) −24.66, (9) −0.099135, (10) −8.9092, (11) 14.686, (12) 6.8994, (13) 6.9794, (14) −10.718, (15) −20.778, (16) 24.483, (17) 12.405, (18) 8.606, (19) 3.4154, (20) 4.7773, (21) 6.4465, (22) 8.1565, (23) 5.1282, (24) −2.2985, (25) −0.56906, (26) 8.835, (27) −6.6331, (28) −18.462, and (29) −35.075. The weight for the hidden neuron 1 and 2 were −1.8299 and −0.12008 respectively. The bias value for the two hidden neuron 1 and 2 were −87.761 and −1.9024 respectively. The bias value for the output neuron was 1.0451.

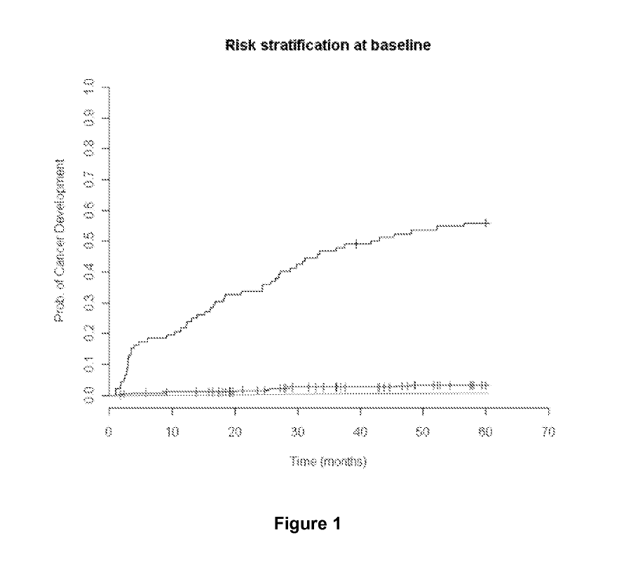

Using the risk prediction model and the 29 risk prediction feature weights presented in Example 5, the EA risk prediction was calculated for the esophageal epithelium biopsy samples. As revealed in FIG. 1, a Kaplan-Meier (KM) curve plot based on the first pre-cancer biopsy samples shows the subjects with samples having an EA risk score s of 0.50 or greater assigned to the high risk group (the top line). The subjects with samples having an EA risk score s of 0.05-0.49 were assigned to the medium risk group (the middle line). The low risk group indicated subjects with samples having an EA risk score s of 0.049 or lower (to bottom line).

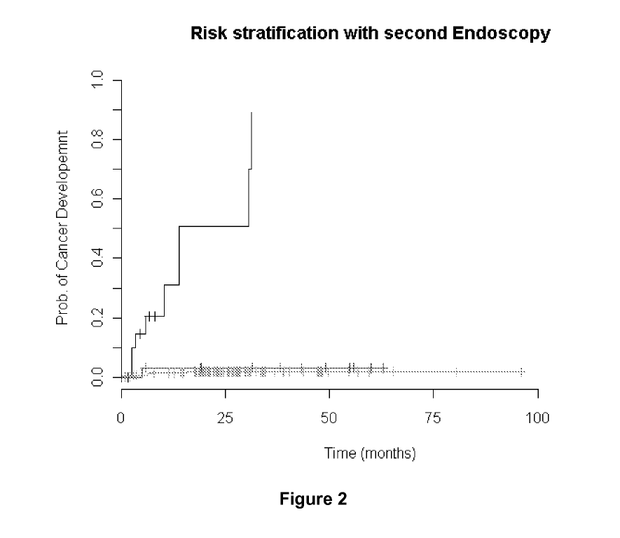

As shown in FIG. 2, a second biopsy (endoscopy) sample was used to further stratify the medium risk group from FIG. 1 into high, medium, and low risk groups. At the time of the second endoscopy, most of the medium risk patents were assigned to the low-risk group; those assigned to the high-risk group were correctly predicted to have a high risk of progression to EA and showed EA within approximately 30-35 months post assessment (top line).

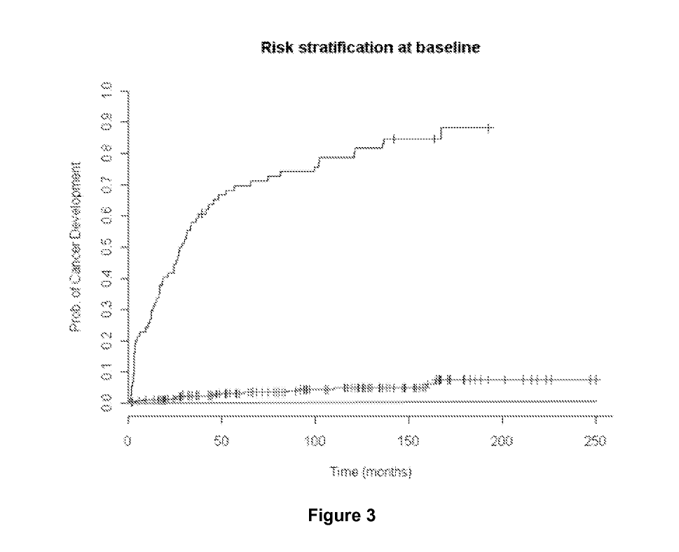

FIG. 3 shows the three EA risk groups sample biopsy data collected over a 250-month interval from baseline assessment (or first biopsy). The cancer risk prediction model disclosed herein was used to stratify subjects into 3 risk groups, high (top line), medium (middle line) and low risk (bottom line). The results show the accuracy of the risk prediction model for correctly detecting and predicting EA risk in a subject.

### Example 6

The EA risk prediction model disclosed herein was used to predict and detect EA risk in subjects with late stage EA. Esophageal epithelium biopsy samples were provided from 6 subjects who had confirmed clinical diagnosis for EA and were not participants in the original study used to develop the risk prediction model. The samples were prepared and examined, for the specific SGA located at the 86 chromosomal regions listed in Table 3, using an Illumina Omni Quad 1M SNP array (Illumina Inc., San Diego, Calif.). The results of the SGA analysis for the 6 subjects are shown in Table 5.

TABLE 5

| | | SGA of EA | SGA of EA | SGA of EA | SGA of EA | SGA of EA | SGA of EA | | Chr. |
|---|---|---|---|---|---|---|---|---|---|
| | | sample 1 | sample 2 | sample 3 | sample 4 | sample 5 | sample 6 | | location |
| | | (0 = no, | (0 = no, | (0 = no, | (0 = no, | (0 = no, | (0 = no, | | (i-1 to i |
| Regions | SGA types | 1 = yes) | 1 = yes) | 1 = yes) | 1 = yes) | 1 = yes) | 1 = yes) | Chr. # | Mb) |
| 1 | gain | 1 | 1 | 0 | 1 | 0 | 1 | 1 | 9-10 |
| 2 | gain | 1 | 0 | 0 | 0 | 0 | 1 | 3 | 178-179 |
| 3 | gain | 1 | 1 | 1 | 0 | 1 | 0 | 6 | 1-2 |
| 4 | gain | 1 | 1 | 1 | 0 | 0 | 0 | 6 | 5-6 |
| 5 | gain | 0 | 1 | 1 | 0 | 1 | 1 | 8 | 32-33 |
| 6 | gain | 0 | 1 | 1 | 0 | 1 | 1 | 8 | 33-34 |
| 7 | gain | 0 | 1 | 0 | 0 | 0 | 0 | 9 | 16-17 |
| 8 | gain | 0 | 1 | 0 | 0 | 0 | 1 | 9 | 72-73 |
| 9 | gain | 0 | 1 | 0 | 0 | 0 | 1 | 9 | 74-75 |
| 10 | gain | 0 | 1 | 1 | 0 | 0 | 1 | 12 | 57-58 |
| 11 | gain | 0 | 1 | 0 | 1 | 1 | 0 | 13 | 44-45 |
| 12 | gain | 0 | 1 | 1 | 1 | 0 | 1 | 13 | 71-72 |
| 13 | gain | 1 | 1 | 0 | 1 | 0 | 1 | 15 | 70-71 |
| 14 | gain | 1 | 1 | 1 | 0 | 1 | 0 | 17 | 37-38 |
| 15 | gain | 1 | 0 | 0 | 1 | 1 | 0 | 18 | 19-20 |
| 16 | gain | 0 | 1 | 0 | 0 | 1 | 0 | 18 | 30-31 |
| 17 | gain | 0 | 0 | 0 | 0 | 0 | 0 | Y | 3-4 |
| 18 | HD | 0 | 0 | 0 | 0 | 0 | 0 | 6 | 1-2 |
| 19 | HD | 0 | 0 | 0 | 0 | 0 | 0 | 9 | 10-11 |
| 20 | HD | 0 | 0 | 0 | 0 | 1 | 0 | 20 | 15-16 |
| 21 | HD | 0 | 0 | 0 | 0 | 0 | 0 | 21 | 36-37 |
| 22 | cnLOH | 0 | 0 | 0 | 0 | 0 | 0 | 1 | 21-22 |
| 23 | cnLOH | 0 | 0 | 0 | 0 | 0 | 0 | 1 | 39-40 |
| 24 | cnLOH | 1 | 0 | 0 | 0 | 0 | 0 | 2 | 202-203 |
| 25 | cnLOH | 1 | 0 | 0 | 0 | 0 | 0 | 2 | 208-209 |
| 26 | cnLOH | 1 | 0 | 0 | 0 | 0 | 0 | 2 | 226-227 |
| 27 | cnLOH | 1 | 0 | 1 | 1 | 0 | 0 | 3 | 41-42 |
| 28 | cnLOH | 0 | 0 | 0 | 1 | 0 | 1 | 4 | 46-47 |
| 29 | cnLOH | 0 | 0 | 0 | 1 | 0 | 1 | 4 | 160-161 |
| 30 | cnLOH | 1 | 0 | 1 | 1 | 0 | 0 | 5 | 84-85 |
| 31 | cnLOH | 1 | 0 | 1 | 1 | 0 | 0 | 5 | 91-92 |
| 32 | cnLOH | 1 | 0 | 1 | 1 | 0 | 0 | 5 | 93-94 |
| 33 | cnLOH | 1 | 0 | 1 | 1 | 0 | 0 | 5 | 121-122 |
| 34 | cnLOH | 1 | 0 | 1 | 1 | 0 | 0 | 5 | 136-137 |
| 35 | cnLOH | 1 | 0 | 1 | 1 | 0 | 0 | 5 | 170-171 |
| 36 | cnLOH | 1 | 0 | 0 | 0 | 0 | 0 | 6 | 29-30 |
| 37 | cnLOH | 1 | 0 | 0 | 0 | 0 | 1 | 6 | 62-63 |
| 38 | cnLOH | 0 | 0 | 0 | 0 | 0 | 1 | 6 | 146-147 |
| 39 | cnLOH | 0 | 0 | 0 | 0 | 0 | 0 | 7 | 66-67 |
| 40 | cnLOH | 1 | 0 | 0 | 0 | 1 | 0 | 7 | 78-79 |
| 41 | cnLOH | 0 | 0 | 1 | 0 | 0 | 0 | 8 | 138-139 |
| 42 | cnLOH | 0 | 0 | 0 | 0 | 0 | 0 | 9 | 134-135 |
| 43 | cnLOH | 0 | 0 | 0 | 1 | 0 | 0 | 10 | 22-23 |
| 44 | cnLOH | 0 | 0 | 0 | 0 | 0 | 0 | 10 | 61-62 |
| 45 | cnLOH | 0 | 0 | 1 | 1 | 0 | 0 | 11 | 18-19 |
| 46 | cnLOH | 0 | 0 | 1 | 1 | 0 | 0 | 11 | 38-39 |
| 47 | cnLOH | 0 | 0 | 1 | 0 | 0 | 0 | 11 | 50-51 |
| 48 | cnLOH | 0 | 0 | 0 | 0 | 0 | 1 | 11 | 110-111 |
| 49 | cnLOH | 0 | 0 | 0 | 0 | 0 | 1 | 12 | 6-7 |
| 50 | cnLOH | 0 | 0 | 0 | 0 | 0 | 0 | 13 | 42-43 |
| 51 | cnLOH | 0 | 0 | 0 | 0 | 1 | 0 | 13 | 58-59 |
| 52 | cnLOH | 0 | 0 | 0 | 0 | 0 | 0 | 14 | 61-62 |
| 53 | cnLOH | 0 | 0 | 0 | 0 | 0 | 0 | 15 | 88-89 |
| 54 | cnLOH | 0 | 0 | 1 | 0 | 0 | 0 | 16 | 61-62 |
| 55 | cnLOH | 0 | 1 | 1 | 0 | 0 | 0 | 16 | 76-77 |
| 56 | cnLOH | 0 | 1 | 1 | 0 | 1 | 1 | 17 | 9-10 |
| 57 | cnLOH | 0 | 1 | 1 | 0 | 1 | 1 | 17 | 12-13 |
| 58 | cnLOH | 0 | 0 | 1 | 0 | 0 | 0 | 19 | 33-34 |
| 59 | cnLOH | 0 | 0 | 1 | 0 | 0 | 0 | 19 | 34-35 |
| 60 | cnLOH | 0 | 0 | 1 | 1 | 0 | 0 | 19 | 48-49 |
| 61 | loss | 0 | 0 | 1 | 0 | 0 | 0 | 1 | 36-37 |
| 62 | loss | 0 | 0 | 0 | 1 | 0 | 0 | 1 | 93-94 |
| 63 | loss | 1 | 1 | 1 | 0 | 1 | 0 | 3 | 60-61 |
| 64 | loss | 0 | 0 | 0 | 1 | 0 | 0 | 4 | 3-4 |
| 65 | loss | 0 | 0 | 0 | 0 | 0 | 1 | 4 | 40-41 |
| 66 | loss | 0 | 0 | 0 | 0 | 0 | 0 | 6 | 62-63 |
| 67 | loss | 0 | 0 | 0 | 0 | 0 | 0 | 7 | 77-78 |
| 68 | loss | 0 | 0 | 0 | 0 | 0 | 0 | 8 | 127-128 |
| 69 | loss | 1 | 0 | 1 | 0 | 0 | 0 | 9 | 0-1 |
| 70 | loss | 1 | 0 | 1 | 0 | 0 | 0 | 9 | 8-9 |
| 71 | loss | 0 | 0 | 1 | 0 | 0 | 0 | 9 | 33-34 |
| 72 | loss | 0 | 0 | 1 | 0 | 0 | 0 | 9 | 35-36 |
| 73 | loss | 0 | 0 | 1 | 0 | 0 | 0 | 9 | 38-39 |
| 74 | loss | 0 | 0 | 0 | 0 | 0 | 0 | 9 | 65-66 |
| 75 | loss | 0 | 0 | 0 | 0 | 1 | 0 | 10 | 25-26 |
| 76 | loss | 0 | 0 | 0 | 0 | 0 | 0 | 10 | 77-78 |
| 77 | loss | 1 | 0 | 0 | 0 | 0 | 0 | 12 | 45-46 |
| 78 | loss | 0 | 0 | 1 | 0 | 1 | 0 | 17 | 8-9 |
| 79 | loss | 0 | 0 | 0 | 0 | 0 | 0 | X | 7-8 |
| 80 | loss | 0 | 0 | 0 | 0 | 0 | 0 | X | 42-43 |
| 81 | loss | 0 | 0 | 0 | 0 | 0 | 0 | X | 154-155 |
| 82 | loss | 0 | 0 | 0 | 0 | 0 | 0 | Y | 8-9 |
| 83 | loss | 1 | 1 | 1 | 1 | 1 | 1 | Y | 13-14 |
| 84 | balanced gain | 0 | 0 | 0 | 0 | 0 | 0 | 1 | 19-20 |
| 85 | balanced gain | 0 | 0 | 0 | 0 | 0 | 0 | 4 | 167-168 |
| 86 | balanced gain | 1 | 0 | 0 | 1 | 0 | 0 | 11 | 55-56 |

The results of the SGA analysis were used to develop a set of 29 risk prediction features according to Table 4. The values for the set of 29 risk prediction features, along with the logistic regression weight values were used in the risk prediction model to calculate a risk score according to formula (3). The risk score was normalized by using formula (2). The risk scores are shown in table 6.

TABLE 6

| | EA Risk Score | |
|---|---|---|
| Subject | (Normalized) | Predicted EA Risk |
| 1 | 1.0 | High |
| 2 | 1.0 | High |
| 3 | 1.0 | High |
| 4 | 1.0 | High |
| 5 | 1.0 | High |
| 6 | 1.0 | High |

The predicted EA risk scores correctly reflect the actual EA disease status in the subjects. Therefore, the results demonstrate the successful use of the EA risk prediction model to correctly predict and detect the risk of EA in a subject.

1. A method of predicting cancer risk in a subject, the method comprising:
obtaining a genetic sample from the subject;
determining the presence or absence of at least one somatic genomic alteration (SGA) in at least one chromosome region from the genetic sample;
selecting at least one risk prediction feature from the at least one chromosomal region;
providing a cancer risk score, wherein the cancer risk score is predictive of the cancer risk in the subject.

2. The method of claim 1, wherein the at least one SGA in the at least one chromosome region comprises at least one of a cnLOH SGA on chromosome 13 between chromosome location 20-115 Mb; a copy number gain SGA on chromosome 15 between chromosome location 20-103 Mb; a copy number gain SGA on chromosome 17 between chromosome location 25-81 Mb; a copy number loss SGA on chromosome 17 between chromosome location 0-23 Mb; a cnLOH SGA on chromosome 17 between chromosome location 0-23 Mb; and a copy number gain SGA on chromosome 18 between chromosome location 0-36 Mb.

3. The method of claim 2, wherein the at least one SGA in the at least one chromosome region further comprises at least one of the SGA listed in Table 3.

4. The method of claim 1, wherein the at least one SGA in the at least one chromosome region is selected from at least one of the SGA listed in Table 3.

5. The method of claim 1, wherein the at least one SGA in the at least one chromosome region comprises the SGA listed in Table 3.

6. The method of claim 1, wherein the at least one risk prediction feature is selected from at least one of the SGA listed in Table 3.

7. The method of claim 1, wherein the at least one risk prediction feature comprises at least one of an allele specific copy gain SGA on chromosome 6 at chromosome location 1-2 Mb; an allele specific copy gain SGA on chromosome 6 at chromosome location 5-6 Mb; an allele specific copy gain SGA on chromosome 15 at chromosome location 70-71 Mb; an allele specific copy gain SGA on chromosome 17 at chromosome location 37-38 Mb; an allele specific copy gain SGA on chromosome 18 at chromosome location 19-20 Mb; a homozygous deletion SGA on chromosome 2 at chromosome location 226-227 Mb; a cnLOH SGA on chromosome 5 at chromosome location 93-94 Mb; a cnLOH SGA on chromosome 6 at chromosome location 29-30 Mb; a cnLOH SGA on chromosome 6 at chromosome location 146-147 Mb; a cnLOH SGA on chromosome 7 at chromosome location 78-79 Mb; a cnLOH SGA on chromosome 8 at chromosome location 138-139 Mb; a cnLOH SGA on chromosome 11 at chromosome location 38-39 Mb; a cnLOH SGA on chromosome 11 at chromosome location 50-51 Mb; a cnLOH SGA on chromosome 11 at chromosome location 110-111 Mb; a cnLOH SGA on chromosome 13 at chromosome location 42-43 Mb; a cnLOH SGA on chromosome 17 at chromosome location 9-10 Mb; a cnLOH SGA on chromosome 17 at chromosome location 12-13 Mb; a cnLOH SGA on chromosome 19 at chromosome location 48-49 Mb; an allele specific copy loss SGA on chromosome 1 at chromosome location 36-37 Mb; an allele specific copy loss SGA on chromosome 9 at chromosome location 0-1 Mb; an allele specific copy loss SGA on chromosome 9 at chromosome location 9-34 Mb; an allele specific copy loss SGA on chromosome 9 at chromosome location 65-66 Mb; an allele specific copy loss SGA on chromosome 12 at chromosome location 45-46 Mb; an allele specific copy loss SGA on chromosome 17 at chromosome location 8-9 Mb; an allele specific copy loss SGA on the X chromosome at chromosome location 42-43 Mb; an allele specific copy loss SGA on the Y chromosome at chromosome location 13-14 Mb; a sum of the results of all the copy loss SGA from the 86 chromosomal regions from Table 3; and a sum of all the SGA analysis results from the panel of 86 chromosomal regions from Table 3.

8. The method of claim 1, wherein selecting at least one risk prediction feature further comprises determining weight values for the at least one risk prediction feature.

9. The method of claim 8, wherein the weight values are determined using a logistic regression model.

10. The method of claim 1, wherein the at least one risk predication feature comprise the set of 29 risk prediction features in Table 4.

11. The method of claim 10, wherein the weight values for each of the set of 29 risk prediction features in Table 4 are (1) 71.533, (2) 38.664, (3) 11.86, (4) 31.81, (5) 0.82257, (6) 54.66, (7) 63.287, (8) 2.0625, (9) 24.666, (10) 101.06, (11) 79.646, (12) 61.317, (13) −291.97, (14) 12.137, (15) 23.348, (16) −70.412, (17) 99.209, (18) 47.058, (19) 109.08, (20) 68.945, (21) −2.394, (22) 1.649, (23) −27.847, (24) 6.6363, (25) −0.078246, (26) 86.339, (27) 1.9427, (28) −0.033952, (29) 0.11415.

12. The method of claim 1, wherein providing a cancer risk score comprises calculating a cancer risk score using the formula (1):
s=β₀+Σᵢ₌₁ⁿβᵢxᵢ (1)
wherein xᵢ is the at least one risk prediction feature from 1 to n, and wherein βᵢ is the weight value assigned to the risk prediction feature xᵢ.

13. The method of claim 1, wherein providing a cancer risk score comprises providing a normalized cancer risk score.

14. The method of claim 1, wherein the genetic sample is obtained from a subject diagnosed with Barrett's esophagus.

15. The method of claim 1, wherein the genetic sample is obtained from a subject having a risk of esophageal adenocarcinoma (EA).

16. The method of claim 1, wherein the genetic sample is obtained from a subject having a risk of esophageal adenocarcinoma (EA), wherein a normalized cancer risk score of approximately 0.50 or greater predicts a high risk of EA in the subject, wherein a normalized cancer risk score of between approximately 0.05 and approximately 0.49 predicts a medium risk of EA in the subject, and wherein a normalized cancer risk score of between approximately 0.00 and approximately 0.049 predicts a low risk of EA in the subject.

17. A method of predicting esophageal adenocarcinoma (EA) risk in a subject, the method comprising:
obtaining a genetic sample from a subject at risk of EA;
determining the presence or absence of at least one somatic genomic alteration (SGA) in at least one chromosome region from the genetic sample, the SGA selected from at least one SGA listed in Table 3;
selecting at least one risk prediction feature from the at least one chromosomal region, wherein the at least one risk prediction feature is selected from at least one of the SGA listed in Table 3;
providing a normalized cancer risk score, wherein a normalized cancer risk score of approximately 0.50 or greater is predictive of a high risk of EA in the subject, wherein a normalized cancer risk score of between approximately 0.05 and approximately 0.49 is predictive of a medium risk of EA in the subject, and wherein a normalized cancer risk score of between approximately 0.00 and approximately 0.049 is predictive of a low risk of EA in the subject.

18. The method of claim 17, wherein the at least one SGA in the at least one chromosome region comprises at least one of a cnLOH SGA on chromosome 13 between chromosome location 20-115 Mb; a copy number gain SGA on chromosome 15 between chromosome location 20-103 Mb; a copy number gain SGA on chromosome 17 between chromosome location 25-81 Mb; a copy number loss SGA on chromosome 17 between chromosome location 0-23 Mb; a cnLOH SGA on chromosome 17 between chromosome location 0-23 Mb; a and copy number gain SGA on chromosome 18 between chromosome location 0-36 Mb.

19. The method of claim 17, wherein selecting at least one risk prediction feature further comprises determining weight values for the at least one risk prediction feature.

20. The method of claim 17, wherein providing a normalized cancer risk score comprises calculating a cancer risk score using the formula (1):
s=β₀+Σᵢ₌₁ⁿβᵢxᵢ (1)
wherein xᵢ is the at least one risk prediction feature from 1 to n;
wherein βᵢ is the weight value assigned to the risk prediction feature xᵢ; and
wherein the calculated risk score (s) may be normalized to a range between 0 and 1 by setting β₀ to −3.108 and then calculating the normalized risk score using formula (2):
1/(1+e^{-s}) (2).
